(19) United States
(12) Patent Application Publication
(10) Pub. No.: US 2009/0259210 A1
(43) Pub. Date: Oct. 15, 2009

(54) METHOD, APPARATUS AND KITS FOR FORMING STRUCTURAL MEMBERS WITHIN THE CARDIAC VENOUS SYSTEM

(76) Inventor: Hani N. Sabbah, Waterford, MI (US)

Correspondence Address: CYR & ASSOCIATES, P.A., 605 U.S. Highway 169, Suite 300, Plymouth, MN, 55441, US

(21) Appl. No.: 12/421,482

(22) Filed: Apr. 9, 2009

Related U.S. Application Data
(60) Provisional application No. 61/123,700, filed on Apr. 10, 2008.

(51) Int. Cl. A61M 25/10

(52) U.S. Cl. 604/509; 604/102.01; 606/191

(57) ABSTRACT

Material may be implanted or injected into cardiac veins as discrete masses for treating various cardiac conditions. A catheter suitable for delivering occluding agent into the cardiac venous system the heart includes a distal end that is positionable within a cardiac vein at a location where an occlusion is to be established. A barrier is provided for occluding the vein adjacent the barrier. A lumen (more than one lumen may be provided if desired) is disposed within the catheter tube and passes through the barrier to terminate at an aperture (more than one aperture may be provided if desired) distal of the barrier, for delivering the occluding agent into the vein beyond the barrier. An inner tube may be provided for introducing an occluder into the vein and spaced away from the barrier, for defining a vein segment between the barrier and the occluder. Methods and kits are also contemplated.

### CROSS-REFERENCE TO RELATED APPLICATIONS

This application claims the benefit of U.S. Provisional Patent Application Ser. No. 61/123,700, filed Apr. 10, 2008, which hereby is incorporated herein in its entirety by reference thereto.

### BACKGROUND OF THE INVENTION

1. Field of the Invention

The present invention relates to treatment of cardiac conditions in living beings, and more particularly to forming structural members within the cardiac venous system for the treatment of cardiac conditions in living beings.

2. Description of Related Art

Cardiovascular disease (“CVD”) is the leading cause of death in the United States; see, e.g., C. Lenfant, Fixing the Failing Heart, Circulation, Vol. 95, 1997, pages 771-772; American Heart Association, Heart and Stroke Statistical Update, 2001; C. Lenfant, Cardiovascular Research: An NIH Perspective, Cardiovasc. Surg., Vol. 5, 1997; pages 4-5; J. N. Cohn et al., Report of the National Heart, Lung, and Blood Institute Special Emphasis Panel on Heart Failure Research, Circulation, Vol. 95, 1997, pages 766-770.

Heart failure (“HF”) is generally defined as a change in the pumping function of the heart accompanied by typical signs or symptoms. Heart failure is a progressive disorder whereby the hemodynamic and symptomatic states of the patient worsen over time despite the absence of clinically apparent adverse events. The symptomatic deterioration is often accompanied by progressive left ventricular (“LV”) chamber remodeling.

Preventing or reversing remodeling has emerged as desirable in the treatment of cardiomyopathy. Cardiomyopathy is a general term for disease of heart muscle regardless of the underlying etiology, which may be, for example, ischemic, hypertensive, dilated, hypertrophic, infiltrative, restrictive, viral, postpartum, valvular, or idiopathic. Cardomyopathy typically results in heart failure.

At the present time, the most effective treatment for patients in end-stage heart failure is heart transplantation. However, given the chronic shortage of donor hearts, alternate strategies are needed to improve the lives of those with heart failure. Moreover, transplantation is not the most suitable treatment option for patients with milder forms of the disease. Other treatment approaches include the delivery of drugs to the site of action through the bloodstream, and the injection of cells into ischemic myocardium to improve cardiac function. An example of an approach for treating cardiovascular problems with intramyocardial scaffolding is disclosed in United States Patent Application Publication No. 2005/0271631, published Dec. 8, 2005 in the name of Lee et al. and entitled “Material compositions and related systems and methods for treating cardiac conditions.” Tissue engineering approaches for cardiac therapy that are generally intended to repair lost or damaged tissue through the use of cellular transplantation and biomaterial scaffolds have also been disclosed. One example of this approach involves suturing fetal cardiomyocyte-seeded alginate gels to the epicardial surface in order to preserve LV function. Another treatment approach involves the use of mechanical external constraints to limit, stop, or even reverse negative left ventricular remodeling. One previously disclosed study included suturing a polymeric mesh to the epicardial surface for the intended purpose of providing an external support to prevent LV dilation and deterioration of LV function post-MI. See Kelley S T, Malekan R, Gorman J H 3rd et al., Restraining infarct expansion preserves left ventricle geometry and function after acute anteroapical infarction, Circulation 1999; 99:135-42. Another previously disclosed device that has been investigated provides a plurality of sutures that are implanted in an open-chest procedure across the ventricle under tension to provide a change in the ventricle shape and a decrease in chamber diameter. This trans-cavitary suture network is intended to decrease the radius of the ventricle to thus reduce ventricular wall stress. Another previously disclosed device under clinical investigation is generally a mesh structure that is implanted as a jacket around the heart and adjusted to provide a snug fit during open-chest surgery. It is intended that the jacket restrains the heart from further enlargement. See, for example, Hani N. Sabbah, Reversal of Chronic Molecular and Cellular Abnormalities Due to Heart Failure by Passive Mechanical Ventricular Containment, Circ. Res., Vol. 93, 2003, pages 1095-1101; Sharad Rastogi et al., Reversal of Maladaptive Gene Program in Left Ventricular Myocardium of Dogs with Heart Failure Following Long-Term Therapy with the Acorn Cardiac Support Devide, Heart Failure Reviews, Vol. 10, 2005, pages 157-163. Still another approach being investigated provides a nitinol mesh as a similar external restraining device to that described above; however, the super-elastic system is intended to assist in systolic contraction, and is generally intended for use via thorascopically guided minimally invasive delivery. Still another system being investigated includes a rigid ring that is implanted during open-chest surgery as another external constraining device to the ventricle. This ring is intended to decrease ventricular wall stress and prevent further enlargement of the heart by reducing the radius and modifying the shape of the ventricle. Examples of devices and methods similar to one or more of those discussed above have been disclosed by various companies, including the following: “Acorn;” “Myocor;” “Paracor;” “Cardioclasp;” and “Hearten.” The Cardioclasp device is disclosed in an article by Abul Kashem et al., CardioClasp: A New Passive Device to Re-Shape Cardiac Enlargement, ASAIO Journal, 2002.

Myocardial infarction (“MI”) is a medical emergency in which some of the heart's blood supply is suddenly and severely reduced or cut off, causing the myocardium to die because it is deprived of its oxygen supply. A myocardial infarction may progressively advance into heart failure. Scar tissue formation and aneurismal thinning of the infarct region often occur in patients who survive myocardial infarctions. It is believed that the death of cardiomyocytes results in negative left ventricular (LV) remodeling which leads to increased wall stress in the remaining viable myocardium. This process results in a sequence of molecular, cellular, and physiological responses which lead to LV dilation. Negative LV remodeling is generally considered an independent contributor to the progression of heart failure.

Mitral regurgitation (“MR”) is incompetency of the mitral valve causing flow from the left ventricle (LV) into the left atrium during systole. Common causes include mitral valve prolapse, ischemic papillary muscle dysfunction, rheumatic fever, and annular dilation secondary to LV systolic dysfunction and dilation.

Despite advances in the treatment of heart failure, aneurismal thinning and mitral regurgitation, further improvement in the speed of treatment and reduction of the complexity and intrusiveness of treatment techniques and devices is desirable. Generally, improved treatment techniques and devices are desirable for the treatment of all forms of cardiomyopathy, including early forms of the disease.

### BRIEF SUMMARY OF THE INVENTION

Each of the various embodiments of the present inventions overcome one or more of the needs and shortcomings discussed above. Additional improvements and advantages may be recognized by those of ordinary skill in the art upon study of the present disclosure.

One embodiment of the invention is an apparatus to form structural members in the cardiac venous system in order to reinforce the myocardium are provided. In various aspects the apparatus may include a catheter tube. The catheter tube defines a distal end, a proximal end, an outer surface, and an inner surface, and the inner surface defines a lumen. The apparatus may include a barrier. In various aspects, the barrier is disposed generally about the distal end of the catheter tube. The barrier is transformable between a collapsed position and an expanded position. The barrier in the collapsed position is deliverable into the vein segment and the barrier in the expanded positions occludes the vein segment. The barrier cooperates with the catheter tube to allow occluding agent to be delivered through the lumen into portions of the vein segment distal of the barrier.

Another embodiment of the invention is a catheter for establishing an occlusion within a cardiac vein, comprising an elongated catheter body having a distal end and a proximal end and comprising an injectate lumen extending longitudinally within the catheter body; a barrier disposed about a periphery of the catheter body in proximity to the distal end thereof and about the injectate lumen, the barrier being controllably transformable between a collapsed position for movement of the catheter body within the cardiac vein, and an expanded position for occluding the cardiac vein in cooperation with the catheter body; and an injectate port disposed in the catheter body distally of the barrier, the injectate lumen being in fluid communication with the injectate port.

Another embodiment of the invention is a catheter for establishing an occlusion within a cardiac vein, comprising means for advancing a distal end of a catheter to a site within the cardiac vein; means for establishing a first venous occlusion about the catheter near a distal end thereof to occlude the cardiac vein at the site, in cooperation with the catheter; means for introducing an occluding agent into the cardiac vein at the site to form at the site a second venous occlusion generally contiguous to the first venous occlusion; and means for withdrawing the distal end of the catheter from the site following the occluding agent introducing step.

Another embodiment of the invention is a method for forming structural members in the cardiac venous system, which includes occluding a vein segment by transforming a barrier disposed about the distal end of a catheter tube from a collapsed position into an expanded position at a vein segment proximal end of the vein segment, and delivering a occluding agent through a lumen defined by the catheter tube into the vein segment distal of the barrier.

Another embodiment of the invention is a method for establishing an occlusion within a segment of a cardiac vein, comprising advancing a distal end of a catheter to a site within the cardiac vein; establishing a first venous occlusion about the catheter in proximity to a distal end thereof to occlude the cardiac vein at the site, in cooperation with the catheter; introducing an occluding agent into the cardiac vein at the site to form at the site a second venous occlusion generally contiguous to the first venous occlusion; and withdrawing the distal end of the catheter from the site following the occluding agent introducing step.

Another embodiment of the invention is a method for establishing an occlusion within a cardiac vein, comprising positioning a distal end of a catheter outer tube within the vein; expanding a barrier disposed about the catheter outer tube near the distal end thereof for occluding the vein at a first location with an expanded barrier, in cooperation with the catheter; positioning a distal end of a catheter inner tube within the vein and spaced apart from the barrier, the distal end of the catheter inner tube having an occluder coupled thereto; expanding the occluder within the vein for occluding the vein with an expanded occluder at a second location spaced-away from the first location; introducing occluding agent from the catheter into the vein between the expanded barrier at the first location and the expanded occluder at the second location; releasing the expanded occluder from the distal end of the inner tube; collapsing the barrier; and withdrawing the catheter from the vein.

Another embodiment of the invention is a heart prosthetic for treating a heart in a diseased condition, comprising a first occlusion of a first composition disposed within a part of a cardiac vein; and a second occlusion of a second composition different than the first composition disposed within a part of the cardiac vein contiguous to the first occlusion; the first and second occlusions being essentially in a solid state for provide structural support to the heart.

Another embodiment of the invention is a kit which, in various aspects, includes an occluding agent and a catheter. The occluding agent solidifies from the liquid state to a solid state within a vein segment to form at least a portion of a structural member. The catheter includes a catheter tube and a barrier. The catheter tube has a proximal end and a distal end and defines a lumen. The barrier is disposed about the distal end and the barrier is transformable between a collapsed position and an expanded position. The catheter is positionable within a vein segment, and the catheter is configured to deliver the occluding agent into portions of the vein segment distal of the barrier.

Another embodiment of the invention is a kit comprising a source of an injectable occluding agent; and a catheter. The catheter comprises an elongated catheter body, a coupler, and an injection port. The catheter body has a distal end and a proximal end and comprising an injectate lumen extending longitudinally within the catheter body, and a barrier disposed about a periphery of the catheter body in proximity to the distal end thereof and about the injectate lumen, the barrier being controllably transformable between a collapsed position for movement of the catheter body within the cardiac vein, and an expanded position for occluding the cardiac vein in cooperation with the catheter body. The coupler is in fluid communication with the injectate lumen, the occluding agent source being adapted for coupling to the first coupler. The injectate port is disposed in the catheter body distally of the barrier, the injectate lumen being in fluid communication with the injectate port.

Other features and advantages of the inventions will become apparent from the following detailed description and from the claims.

### BRIEF DESCRIPTION OF THE SEVERAL VIEWS OF THE DRAWINGS

FIG. 1 illustrates in perspective view an exemplary implementation of a catheter.

FIG. 2A illustrates in perspective view an exploded distal portion of the catheter of FIG. 1 in a first operating condition.

FIG. 2B illustrates in perspective view an exploded distal portion of the catheter of FIG. 1 in a second operating condition.

FIG. 2C illustrates in perspective view an exploded distal portion of the catheter of FIG. 1 in a third operating condition.

FIG. 2D illustrates in a cross-sectional view a distal portion of the catheter of FIG. 1.

FIG. 2E illustrates in a cross-sectional view another distal portion of the catheter of FIG. 1.

FIG. 3 illustrates in a cross-sectional view a distal portion of the catheter of FIG. 1.

FIG. 4A is a schematic illustration of a catheter in a first state of deployment.

FIG. 4B is a schematic illustration of a catheter in a second state of deployment.

FIG. 4C is a schematic illustration of a catheter in a third state of deployment.

FIG. 4D is a schematic illustration of a catheter in a fourth state of deployment.

FIG. 4E is a schematic illustration of a catheter in a fifth state of deployment.

FIG. 4F is a schematic illustration of a catheter in a sixth state of deployment.

FIG. 4G is a schematic illustration of a catheter in a seventh state of deployment.

FIG. 4H is a schematic illustration of a catheter in a eighth state of deployment.

FIG. 4I is a schematic illustration of a catheter in a ninth state of deployment.

FIG. 4J is a schematic illustration of a catheter in a tenth state of deployment.

FIG. 5 illustrates in a cross-sectional view one example of a structural member that resides in a vein.

FIG. 6 illustrates in a cross-sectional view another example of a structural member that resides in a vein.

FIG. 7 illustrates in a cross-sectional view yet another example of a structural member that resides in a vein.

FIG. 8A illustrates in a cross-sectional view a distal portion of an exemplary catheter having a barrier in a collapsed position.

FIG. 8B illustrates in a cross-sectional view the catheter of FIG. 10A with the barrier in an expanded position.

FIG. 9A illustrates in perspective view a distal portion of an exemplary embodiment of a catheter in a first operating condition.

FIG. 9B illustrates in perspective view the catheter of FIG. 9B in a second operating condition.

The Figures are to facilitate explanation of the present invention. The number, position, relationship and dimensions of the parts shown in the Figures to form the various implementations described herein, as well as dimensions and dimensional proportions to conform to specific force, weight, strength, flow and similar requirements, are explained herein or are understandable to a person of ordinary skill in the art upon reading this patent. Where used in various Figures, the same numerals designate the same or similar parts. Furthermore, when the terms “top,” “bottom,” “right,” “left,” “forward,” “rear,” “first,” “second,” “inside,” “outside,” and similar terms are used, the terms should be understood in reference to the orientation of the structures shown in the drawings and utilized to facilitate understanding. Similarly, when the terms “proximal,” “distal,” and similar positional terms are used, the terms should be understood in reference to the structures shown in the drawings and utilized to facilitate understanding.

### DETAILED DESCRIPTION OF THE INVENTION, INCLUDING THE BEST MODE

Occluding agent may be delivered into one or more selected sections of the cardiac venous system by a catheter to form one or more structural members configured to reinforce the myocardium in order to prevent, moderate, stop, or reverse negative cardiac remodeling due to various adverse cardiac conditions, both acute and chronic. The cardiac conditions that may be treated using the apparatus and methods described herein may include cardiomyopathy, myocardial infarctions, acute myocardial infarctions, arrhythmias, valvular insufficiency, congestive heart failure, mitral regurgitation and other heart valve abnormalities, other cardiac complications, and combinations thereof. Kits for treating the cardiac conditions using the methods described herein are also contemplated.

The myocardium is composed of interlacing bundles of cardiac muscle fibers arranged spirally around the circumference of the heart. These cardiac muscle fibers receive blood through coronary circulation. The coronary arteries branch from the aorta just beyond the aortic valve to supply blood to the cardiac muscle fibers, and the coronary veins empty into the right atrium via the coronary sinus. The myocardium is well supplied with a highly distributed system of coronary arteries and veins. Typically, each coronary artery as it courses along the surface of the heart has coronary veins that course generally alongside. This is also generally true of the smaller branches of the main coronary arteries, including those that penetrate into the myocardium and perfuse the deeper layers of the muscle of the heart. Small veins such as venules return the blood to larger cardiac veins. Thus the venous system network of the heart is distributed throughout the thickness of the heart muscle and is present everywhere arteries are present.

Material may be implanted or injected into cardiac veins as discrete masses at various sites in the cardiac venous system, where it occludes the vein at the site of injection but also disperses in the vein and also into venules and possibly even the capillaries in fluid communication with the site of injection in order to reinforce the myocardium for the purpose of preventing, moderating, stopping or reversing negative cardiac remodeling due to various adverse cardiac conditions, both acute and chronic, or for the purpose of treating localize anomalies of the heart, or for both purposes. Cardiac conditions that may be treated using such techniques include cardiomyopathy, myocardial infarctions, acute myocardial infarctions, arrhythmias, valvular insufficiency, congestive heart failure, mitral regurgitation and other heart valve abnormalities, and other cardiac complications. Kits for treating the cardiac conditions using the techniques described herein are also contemplated. Exemplary techniques are described in US Patent Application Publication No. US 2008/0269720, published Oct. 30, 2008 (Sabbah, “Cardiac Repair, Resizing and Reshaping Using the Venous System of the Heart”), which hereby is incorporated herein in its entirety by reference thereto.

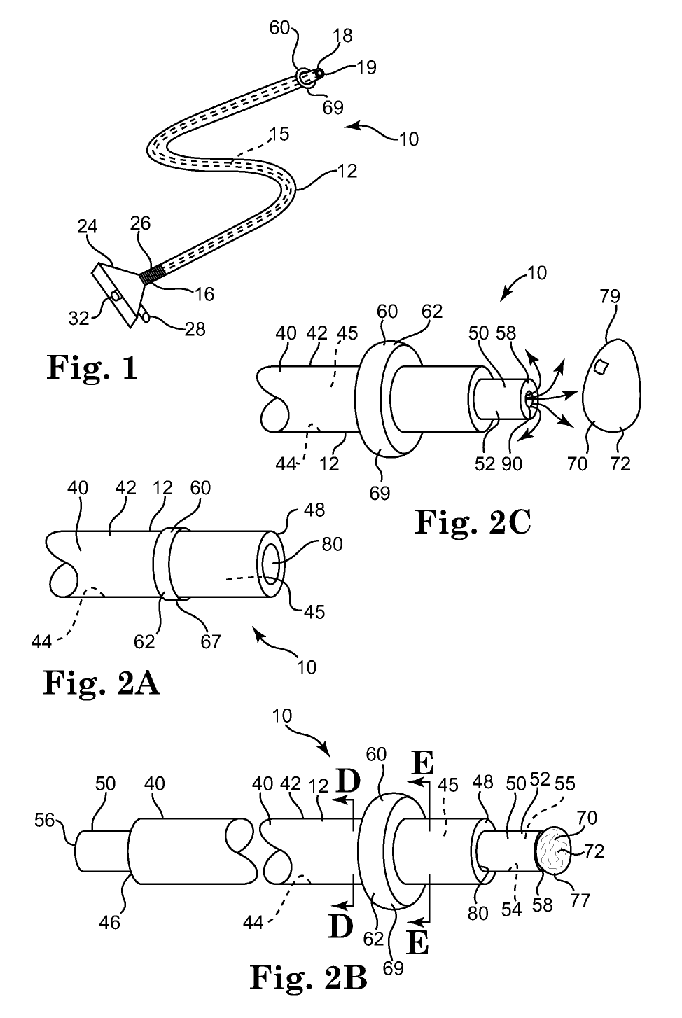

FIG. 1 shows a catheter 10 that is particularly advantageous for delivering occluding agent into the venous system of the heart. In various aspects, occluding agent is delivered into various vein segments by the catheter 10 to form structural members. The vein segments, in various aspects, may include a major part of a cardiac vein, or may include small portions of a cardiac vein such as a distal portion, a proximal portion, or a medial portion. In various aspects, occluding agent may be disposed in a series of segments within a cardiac vein, thereby forming a series of structural members within the particular cardiac vein. The occluding agent, in various aspects, may be disposed in vein segments within several cardiac veins and may be disposed within several segments of the several cardiac veins.

Although one or more vein segments of the cardiac venous system are occluded by the structural members formed from the occluding agent, occlusion of the vein segments is not adverse to treatment. Veins have much thinner walls with less smooth muscle than arteries. Relative to arteries, veins have very little elasticity because venous connective tissue contains considerably more collagen fibers than elastin fibers. Moreover, venous smooth muscle has little inherent myogenic tone. Accordingly, veins are highly distensible and have little elastic recoil, so that non-occluded veins in proximity to occluded veins can easily distend to accommodate additional volumes of blood diverted from the occluded vein segment 400 with only a small increase in venous pressure.

The occluding agent may be delivered into vein segments of the cardiac venous system by the catheter 10 to form structural members configured to treat a localized heart anomaly, the heart generally, the ventricle(s), or the atria. Where a generalized treatment is desired, mapping need not be performed to select the sections into which the occluding agent is delivered. A generalized approach is particularly applicable to the ventricles. Where a localized treatment is desired, the site of the heart disorder such as a myocardial infract may be identified, and vein segments of the cardiac venous system encompassing the localized heart disorder may be selected. Occluding agent may be delivered by the catheter 10 into the vein segments to form structural members to reshape and/or remodel the atria, and in particular an enlarged left atrium, and/or to aid in prevention of atrial fibrillation and/or other atria-related conditions. Suitable techniques for identifying various heart disorders such as thin walled regions or aneurysms requiring treatment may include MRI, echocaridogram, and other imaging and mapping modalities as would be recognized by those of ordinary skill in the art upon study of this disclosure.

Identifying the vein segments of the cardiac venous system into which the occluding agent may be delivered to form structural members may be done empirically. Alternatively, computer-aided selection may be practiced if desired. In one technique, finite element model simulation is used to model a region of the heart such as the left ventricle. For example, using an imaging or mapping technique, parameters of the patient's left ventricle, including the location, extent and thickness of damaged wall areas, are measured and added to the model. The formation of structural members in selected vein segments of the cardiac venous system may be simulated by changing the transmural coordinates of epicardial and endocardial mesh nodes in border zone elements corresponding to the selected segments, along with changing the contractility of the elements. The selected vein segments may be changed over successive simulations to identify an optimal set of vein segments of the cardiac venous system to receive occluding agent for the formation of structural members. A suitable finite element model simulation is disclosed in an article by Samuel T. Wall et al., Theoretical Impact of the Injection of Material Into the Myocardium: A Finite Element Model Simulation, in Circulation AHA 106.657270, Nov. 27, 2006, which hereby is incorporated herein in its entirety by reference thereto.

While some cardiac conditions may be treated in one procedure, other cardiac conditions may be treated by successive deliveries of the occluding agent into the cardiac venous system over time. For example, in some aspects, the occluding agent may be delivered into one or more vein segments of the cardiac venous system to form structural member(s), and the effect studied before delivery of the occluding agent into additional vein segment(s) of the cardiac venous system to form additional structural member(s). In various aspects, the delivery of the occluding agent into one or more vein segment(s) of the cardiac venous system to form structural member(s) may be configured to fine tune the beneficial results of prior deliveries.

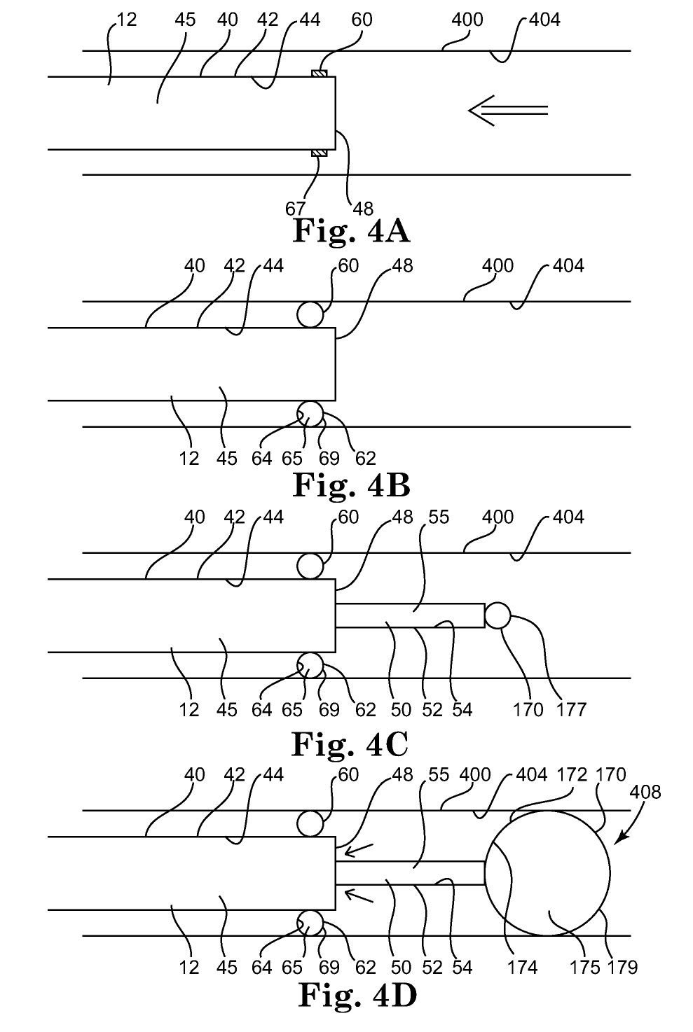

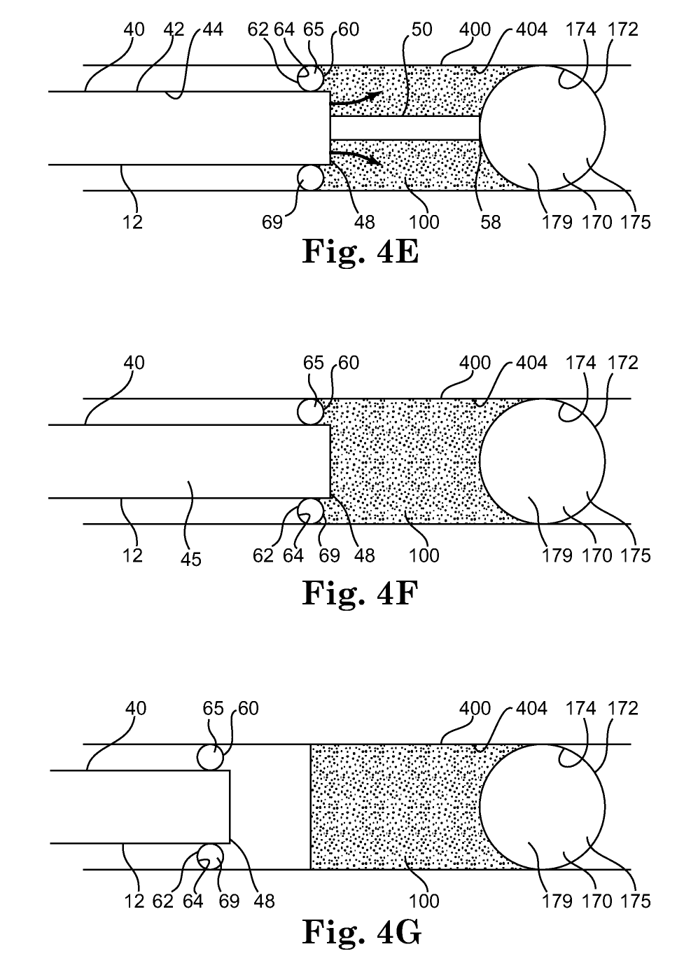

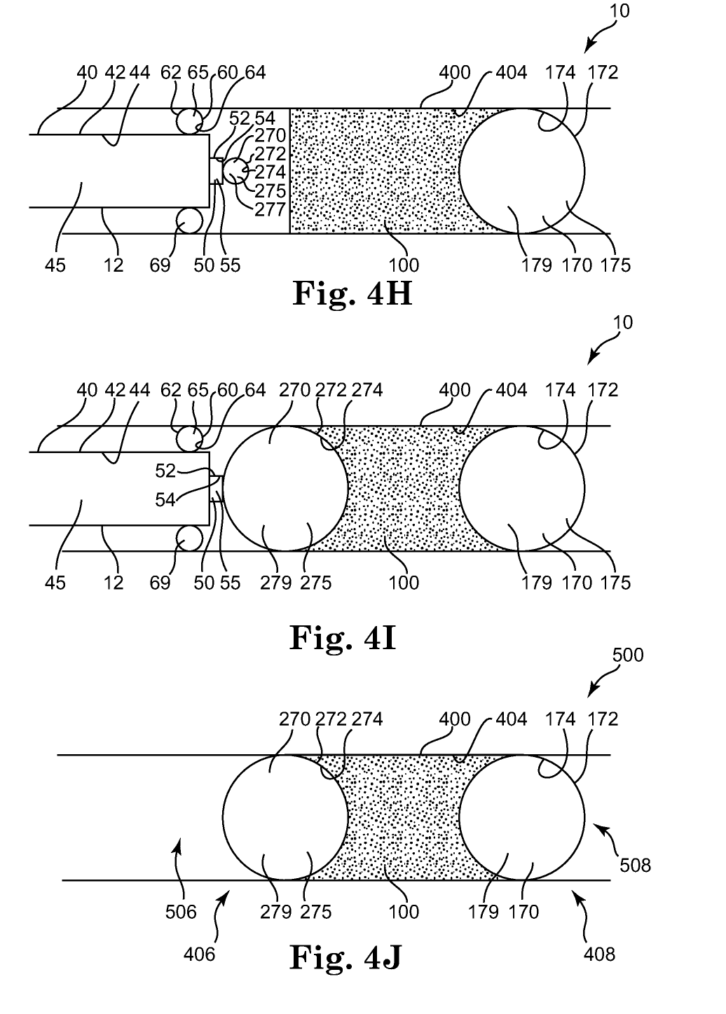

The occluding agent 100 (see FIGS. 4E-4J and 5-7) is suitable for forming a rigid or semi-rigid structural member 500 (see FIGS. 4J and 5-7) within sections of the cardiac venous system to support the wall of the heart in such a way as to prevent, moderate, stop or reverse negative cardiac remodeling. A suitable occluding agent 100 is one that may be delivered in a low viscosity liquid state into one or more vein segments 400 of the cardiac venous system, and that would solidify into a rigid or semi-rigid solid state (including gel) to support the wall of the heart. Accordingly, the occluding agent 100 is transformable between at least two states, a liquid state and a solid state, and may solidify from the liquid state into the solid state. In the liquid state, the occluding agent 100 may pass through the lumen 15 of the catheter 10 into the vein segment 400 and may flow throughout the vein segment 400 to occupy the vein segment 400. The occluding agent 100 may solidify from the liquid state into the solid state, and, in the solid state, the occluding agent 100 may form at least a portion of the structural member 500 to reinforce the myocardium in order to prevent, moderate, stop, or reverse negative cardiac remodeling.

Exemplary occluding agent 100 includes natural and synthetic polymers (any FDA approved polymer for human implantation), fibrin sealants, alginates, collagens, sugars, hydrogels, self-assembling peptides, PLGA, PEG, coagulation protein based sealants, hyaluronic acid, alginate and chitosan hydrogels and beads, alginate material with covalently attached peptides, alginate beads coated with chitosan material, self-assembling peptide scaffold hydrogels, and so forth, either alone or in combinations of two or more. Suitable biopolymer materials are commercially available from a variety of commercial sources, including the NovaMatrix Unit of FMC Biopolymer Corporation, 1735 Market Street, Philadelphia, Pa. 19103 and Omrix Biopharmaceuticals, 630 5th Avenue, 22nd Floor, New York, N.Y. 10111. This list of occluding agent 100 is illustrative, and the occluding agent 100 may be essentially any FDA approved material that has a degree of purity, preparation time, ease of expression (viscosity), reaction rate (cure time), strength (energy to failure), compliance, water uptake, burst strength, tissue adherence, endurance, degradation rate, and so forth, suitable for forming a supportive structure upon delivery into the selected vein segment 400. The various properties of the occluding agent 100 such as stiffness, compliance, and resorption rate may be tailored to the particular condition(s) being treated and may be tailored for the size of the vein segment 400 into which the agent is to be delivered.

The occluding agent 100 may serve as a platform for delivery of other therapeutic materials, including living cells (including, for example, myocytes, fibroblasts, fibrocytes or profibrotic blood progenitor cells, stem cells, and muscle cells), growth factor (including, for example, angiogenic factors such as VEGF, FGF, and HGF; chemotractants; stem cell derived factor; and TGF-b), stem cell products, peptides, proteins, genes, chondrocytes, insoluble molecules, other biologics, and so forth, alone or in combinations of two or more.

The catheter 10, in various aspects, may be used to deliver the occluding agent 100 into the vein segment 400 and, in various aspects, may be configured to prevent entrainment of the occluding agent 100 in the venous flow and subsequent conveyance of the occluding agent 100 into the right atrium of the heart. An implementation of the catheter 10 is illustrated in FIG. 1. The catheter 10 includes catheter tube 12 with distal end 18 and proximal end 16. The distal end 18 is configured to be positionable within the vein segment 400 of the cardiac venous system at which the occluding agent 100 is to be delivered. A lumen 15 is disposed within the catheter tube 12 and terminates at an aperture 19 at the distal end 18 to deliver the occluding agent 100 into the vein segment 400. If a multiple-component occluding agent is used, multiple lumen may be provided that terminate in respective apertures or, if desired, in a mixing chamber that communicates with an aperture. The barrier 60 is secured circumferentially about the distal end 18 of the catheter tube 12. Radiopaque materials may be disposed proximate the distal end 18 in various implementations in order to facilitate navigation of the distal end 18 through the various bodily passages to the vein segment 400 by the physician, and the distal end 18 is atraumatic to avoid damaging the various bodily passages including the vein segment 400.

FIG. 1 also shows that the proximal end 16 of the catheter tube 12 may terminate at a hub 24. The hub 24 and adjacent portions of the catheter tube 12 may include a strain relief 26 configured to relieve torsional strain. The hub 24 may include features through which, inter alia, a guide wire may be inserted and/or extracted and through which fluids including the occluding agent 100 in the liquid state may be communicated to the one or more lumen 15, and the hub 24 is otherwise generally configured to cooperate with the proximal end 16 of the catheter tube 12 as would be understood by one of ordinary skill in the art upon study of this disclosure. For example, the hub 24 may include an inflation port 28 having a coupling, such as a luer-lock type fitting, for connecting one or more of the lumen 15 defined by the catheter 10 to a source of fluid. In various implementations, the hub 24 may include a guidewire port 32. The guidewire port 32 may be in communication with one of the one or more lumen 15 defined by the catheter 10 to receive the guidewire over which the catheter 10 may be passed in order to deliver the catheter 10 into position within the cardiac vein of a patient. The guidewire port 32 may include a hemostatic valve, which allows the guidewire to traverse and slide within the lumen 15 while resisting the flow of blood or other fluids through the lumen 15 and guidewire port 32.

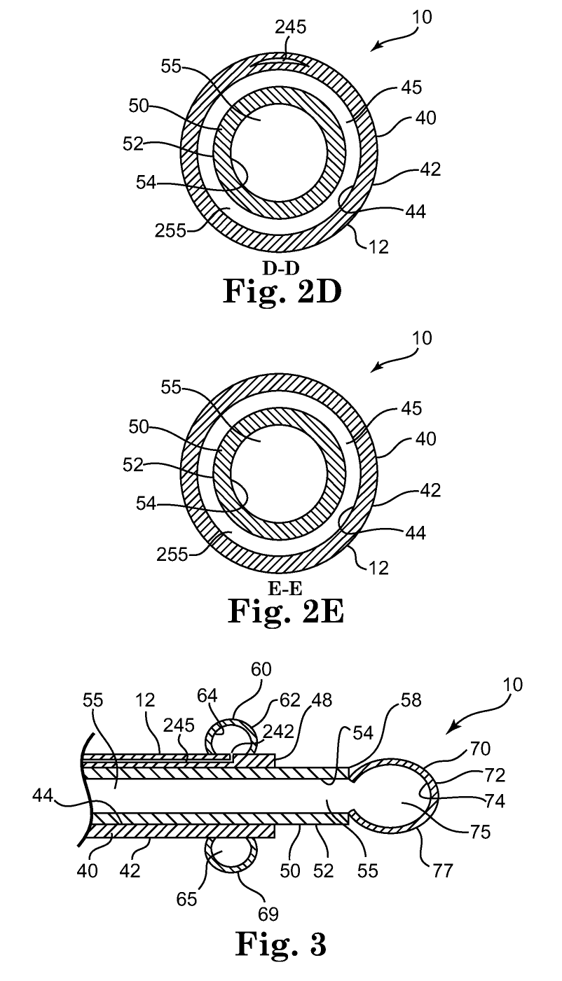

FIGS. 2A, 2B, 2C, 2D, and 2E illustrate an implementation of portions of the catheter 10 in various operating conditions. This illustrated implementation of the catheter 10 includes the catheter tube 12 formed as an outer tube 40 and an inner tube 50. The outer tube 40 defines an outer tube outer surface 42 and an outer tube inner surface 44. The outer tube inner surface 44 defines an outer lumen 45 that passes generally from an outer tube proximal end 46 to an outer tube distal end 48 to communicate fluid and/or to communicate the inner tube 50 generally from the outer tube proximal end 46 to the outer tube distal end 48.

The outer tube 40 may be made from a range of materials. For example, in one implementation, the outer tube 40 may be a metal, such as, for example, stainless steel or nitinol. In another implementation, the outer tube 40 can be made from one or more polymers such as polyethylene, nylon, polyimide, among others. Combinations of materials such as the above materials may also be employed, and the material(s) may be varied along the length of the outer tube 40. The materials and dimensions are generally selected to provide a desired balance of longitudinal stiffness and torsional rigidity based on the characteristics of the outer tube 40 to allow the outer tube 40 to be positioned within the vein segment 400 of the patient.

As illustrated, the barrier 60 extends circumferentially around the outer tube outer surface 42 generally proximate the outer tube distal end 48 to block blood flow between the outer tube outer surface 42 and the vein inner surface 404 (see FIGS. 4A-4J). The barrier 60, as illustrated, defines a barrier outer surface 62 and a barrier inner surface 64, and the barrier inner surface 64 defines a barrier chamber 65 (FIG. 3). The barrier 60 may be constructed of a variety of different materials, including, for example, Nylon, PEEK, Pebax, among others. Portions of the barrier outer surface 62 are engaged with the outer tube outer surface 42 to prohibit the flow of blood between the barrier outer surface 62 and the outer tube outer surface 42.

The barrier 60 is transformable between at least a collapsed position 67 as illustrated in FIG. 2A, and an expanded position 69 as illustrated in FIG. 2B. In the illustrated implementation, the outer tube 40 defines a barrier lumen 245 (FIG. 3) that extends along at least a portion of the outer tube 40 to transmit fluid to/from the barrier 60 in order to inflate/deflate the barrier. The barrier lumen 245, as illustrated in FIG. 2D, has a crescent shape to maximize the flow cross-section. In other implementations, the barrier lumen 245 may have a circular cross-section or other cross-sectional shape, and a plurality of barrier lumen could be provided in various implementations.

With the barrier 60 in the collapsed position 67, the outer tube 40 may be positioned within the vein segment 400 such that the outer tube distal end 48 is generally at the vein segment proximal end 406 (FIG. 4J). Fluid may be communicated via the barrier lumen 245 into the barrier chamber 65 through a barrier port 242 (FIG. 3) to expand the barrier 60 into an expanded position (expanded position 69 is shown in the illustrated implementation of FIG. 3). In other implementations, the barrier 60 may be mechanically expanded such as by the application of an electrical current to a shape memory alloy, or may be otherwise configured to be transformable between at least a collapsed position 67 and in an expanded position 69 in ways recognizable by those of ordinary skill in the art upon study of this disclosure.

In the expanded position 69, portions of the barrier outer surface 62 may be generally biased against the venous inner surface to anchor the outer tube 40 to the venous inner surface 404 and to prevent blood flow from passing between the venous inner surface 404 and the outer tube outer surface 42 in the proximal direction. With the barrier 60 in the expanded position 69, fluid may be communicated from the barrier chamber 65 into the outer tube outer lumen 45 to collapse the barrier 60 into the collapsed position 67 in order to release the barrier outer surface 62 from the venous inner surface 404 so that the outer tube 40 may be withdrawn.

In the implementation of the catheter 10 illustrated in FIGS. 2A and 2B, an inner tube 50 may be slidably received within the outer lumen 45 of the outer tube 40. The inner tube 50 is configured to emplace one or more occluders 70 within the vein segment 400. As illustrated, the inner tube 50 defines an inner tube outer surface 52 and an inner tube inner surface 54. In this implementation, the inner tube inner surface 54 defines an inner lumen 55 that passes generally from an inner tube proximal end 56 to an inner tube distal end 58 to communicate fluid generally from the inner tube proximal end 56 to the inner tube distal end 58. The inner tube 50 may be made from a variety of materials, such as, for example, stainless steel, nitinol, or one or more polymers such as polyethylene, nylon, polyimide, among others, or combinations thereof. The materials are generally selected to allow the inner tube 50 to be advanced and withdrawn through the outer lumen 45 and to communicate fluid, which may be under pressure, through the inner lumen 55.

As illustrated, portions of the inner tube 50 generally proximate the inner tube distal end 58 may extend forth from the outer aperture 80 defined by the terminus of the outer lumen 45 at the outer tube distal end 48, as illustrated in FIG. 2B. In various implementations, radiopaque materials may be disposed proximate the outer tube distal end 48 and/or the inner tube distal end 58 in order to facilitate manipulation of the outer tube 40 and/or the inner tube 50 by the physician. The outer tube distal end 48 and the inner tube distal end 58 are generally configured to be atraumatic in order to avoid damage to the vein segment and other bodily passages as the outer tube distal end 48 and the inner tube distal end 58 are navigated into position by the physician.

The outer tube proximal end 46 and the inner tube proximal end 56 are generally illustrated in FIG. 2B. As illustrated, the inner tube proximal end 56 extends proximally from the outer tube proximal end 46 so that a physician may grasp portions of the outer tube 40 distal to the outer tube proximal end 46 and portions of the inner tube 50 distal to the inner tube proximal end 56 in order to manipulate the outer tube 40 and the inner tube 50 with respect to one another. In various implementations, the outer tube proximal end 46 and the inner tube proximal end 56 may cooperate with the hub 24 to allow the outer tube 40 and the inner tube 50 to be manipulated with respect to one another.

An occluder 70 may be removably disposed upon the inner tube distal end 58, as illustrated in FIGS. 2B and 2C. The occluder 70 may be a balloon having an occluder outer surface 72 and an occluder inner surface 74, and an occluder chamber 75 may be defined by the occluder inner surface 74 (FIG. 3). Many different compliant or semi-compliant material are suitable for a balloon occluder, including, for example, nylon, polyamines, ethylene-vinyl acetate, polyvinyl chloride, olefin copolymers or homopolymers, polyethylenes, polyurethanes, and various blends of polymers and copolymers. Many different materials are suitable for expanding a balloon occluder by filling it, including, for example, hydrogels, silicones and epoxy, and various bioabsorbable materials such as hyaluronic acid injectable fillers, human collagens, and human fibrin sealant. Alternatively, the occluder 70 may be made of a suitable sponge material such as collagen that expands automatically when released into the vein, such materials being known to a person of ordinary skill in the art.

Where the occluder 70 is a balloon-type occluder, it may be inflatable between at least a contracted position 77 and a dilated position 79, and the inner lumen 55 may fluidly communicate with the occluder chamber 75 to introduce fluid into the occluder chamber 75 in order to inflate the occluder 70 from the contracted position 77 into the dilated position 79. With the occluder 70 in the contracted position 77, the occluder 70 may be advanced upon the inner tube distal end 58 through the outer lumen 45. The inner tube distal end 58 may be extended forth from the outer aperture 80 and manipulated to position the occluder 70 within the vein segment 400, and fluid may be communicated from the inner lumen 55 into the occluder chamber 75 to inflate the occluder 70 from the contracted position 77 into the dilated position 79, as illustrated in FIGS. 2B, 2C, and 3. In some implementations, occluding agent 100 may be communicated into the occluder chamber 75 to inflate the occluder 70 into the dilated position 79. In the dilated position 79 illustrated in FIG. 2C, portions of the occluder outer surface 72 may be generally biased against the interior venous surface to substantially block blood flow through the vein segment 400.

The occluder 70 illustrated in FIGS. 2B and 2C has a generally spherical shape in both the contracted position 77 and in the dilated position 79. In other implementations, the occluder 70 may be configured in a variety of shapes, and the shapes may vary between the contracted position 77 and the dilated position. In other implementations, the occluder may be configured to spring mechanically from the contracted position 77 into the dilated position, or may configured to be alterable between at least the contracted position 77 and the dilated position 79 in ways recognizable by those of ordinary skill in the art upon study of this disclosure.

As illustrated in FIG. 2C, the inner tube 50 may be disconnected from the occluder 70, which is in the dilated position 79, and the inner tube distal end 58 at least partially withdrawn toward the outer tube distal end 48. Occluding agent 100 may then be delivered through an inner aperture 90, which is defined by the terminus of the inner lumen 55 at the inner tube distal end 58. In various implementations, the inner tube 50 may be traversed between the occluder 70 and the outer tube distal end 48 as the occluding agent 100 is delivered through the inner aperture 90. In various other implementations, a plurality of apertures could be disposed generally about the inner tube distal end 58 through which the occluding agent 100 could be delivered from the inner lumen 55 into the vein segment 400. In various other implementations, the inner tube 50 may be withdrawn entire from the outer lumen 45 and the occluding agent 100 delivered through, for example, the outer lumen 45 through the outer aperture 80 and into the vein segment 400.

FIG. 2D illustrates a cross-section of the catheter tube 12 proximal of the barrier 60 that includes the crescent-shaped barrier lumen 245. The inner tube 50 is disposed within the outer lumen 45 such that the outer tube inner surface 44 and the inner tube outer surface 52 define an annular lumen 255 for the communication of fluid.

FIG. 2E illustrates a cross-section of the catheter tube 12 distal of the barrier 60. The crescent-shaped barrier lumen 245 is not present in this view, having terminated at the barrier port 247 proximate the barrier (FIG. 3). The annular lumen 255 extends to the distal end 18 of the catheter tube 12 to communicate fluid through the distal end of the catheter tube 12.

Operation of an implementation of the catheter 10 to deliver the occluding agent 100 into the vein segment 400 is generally illustrated in FIGS. 4A to 4J. As illustrated in FIG. 4A, the catheter 10 may be navigated through various bodily passages to place the outer tube distal end 48 into position within the vein segment 400. The barrier 60 is in the collapsed position 67 in FIG. 4A, and blood may flow in the direction indicated between the outer tube outer surface 42 and the vein inner surface 404.

In FIG. 4B, the barrier 60 has been transformed from the collapsed position 67 into the expanded position 69. Portions of the barrier outer surface 62 may bias against the vein inner surface 404 to occlude the vein and to anchor the outer tube 40 within the vein segment 400.

As illustrated in FIG. 4C, the inner tube 50 is passed within the outer lumen 45 and through the outer aperture 80 into the vein segment 400. An occluder 170 in the contracted position 177 is secured to the inner tube distal end 58, as illustrated.

As illustrated in FIG. 4D, the occluder 170 in an expanded condition defines an occluder outer surface, an occluder inner surface 174, and an occluder chamber 175. The occluder 170 is operable to inflate between at least a contracted position 177 (FIG. 4C) and a dilated position 179. The inner tube 50 may be manipulated to position the occluder 170 within the vein segment 400. In various implementations, portions of the occluder 170 and/or portions of the inner tube 50 proximate the inner tube distal end 58 may be coated with various radiopaque materials or otherwise adapted to facilitate placement by the physician. When inflated into the dilated position 179, the occluder 170 generally biases portions of the occluder outer surface against the vein inner surface 404 to occlude the vein and to lock the occluder 170 into the vein segment 400. In some implementations, a gas may be communicated into the occluder chamber 175 to inflate the occluder 170. In other implementations, a liquid such as saline solution may be communicated into the occluder chamber 175 to inflate the occluder 170. In other implementations, the occluding agent 100 may communicated into the occluder chamber 175 to inflate the occluder 170.

As illustrated in FIGS. 4C and 4D, the occluder 170 in the contracted position 177 is positioned proximate the vein segment distal end 408 of the vein segment 400 and then the occluder 170 is inflated into the dilated position 179 to engage the vein inner surface 404 in order to generally occlude the vein segment 400 at the vein segment distal end 408. Blood may then be removed from the vein segment 400 between the occluder 170 and the barrier 60 by being withdrawn through the annular lumen 255 (FIG. 2D), as indicated in FIG. 4C. In other implementations (not shown), the inner tube 50 may be detached from the occluder 170 in the dilated position 179 and the blood may be removed by being withdrawn from the vein segment 400 through the inner aperture 90 (FIG. 2C) and the inner lumen 55. In still other implementations (not shown), the occluder 170 may be positioned proximate the outer tube distal end 48, inflated into the dilated position 179 to engage the vein inner surface 404, and then pushed to the vein segment distal end 408 to force blood out of the vein segment 400 between the barrier 60 and the vein segment distal end 408. In still other implementations, blood may be permitted to remain in the vein segment 400 and is displaced and pushed back into the venules as the occluding agent 100 is introduced and flows into the vein and venules.

In FIG. 4E, occluding agent 100 is delivered into the vein segment 400 through an outer aperture 80 (FIG. 2A). The occluder 170 and the barrier 60 hold the occluding agent 100 within the vein segment 400 while the occluding agent 100 is admitted into the vein segment 400 through the outer aperture 80 after passing through the annular lumen 255 (FIG. 2E). The inner tube 50 remains secured to the occluder 170 to lock the occluder 170 into the vein segment 400 in the illustrated implementation. In other implementations, the inner tube 50 may be detached from the occluder 170, and the occluding agent 100 passed through the inner lumen 55 into the vein segment 400 through the inner aperture 90. In still other implementations, after the inner tube 50 is detached from the occluder 170, the inner tube 50 is substantially withdrawn from the outer lumen 45, and the occluding agent 100 is passed into the vein segment 400 through the outer aperture 80. In still other implementations, the occluding agent 100 may be passed through another lumen within the inner tube 50 into the vein segment 400 through one or more apertures in the wall of the inner tube 50, with the occluder 170 either attached or detached. In still other implementations, the separate components of a multi-component occluding agent may be introduced using a combination of the aforementioned techniques.

As illustrated in FIG. 4F, the inner tube distal end 58 is detached from the occluder 170 and withdrawn from the vein segment 400. In various implementations, the inner tube 50 may be substantially or entirely withdrawn from the outer lumen 45. The barrier 60 remains in the expanded position 69 to secure the occluding agent 100 in the vein segment 400. The occluding agent 100 may be in the liquid state, may be solidifying into the solid state, or may be generally solidified into the solid state.

As illustrated in FIG. 4G, the outer tube distal end 48 with the barrier 60 in the expanded position 69 is withdrawn somewhat in the proximal direction. As illustrated, the occluding agent 100 is at least partly solidified into the solid state so that the occluding agent 100 remains positioned in the vein segment 400.

As illustrated in FIG. 4H, the inner tube 50 is passed through the outer lumen 45 and extends forth from the outer aperture 80 (FIG. 2A). As illustrated, occluder 270 is secured to the inner tube distal end 58 (FIG. 2B). The occluder 270 defines an occluder outer surface 272, an occluder inner surface 274, and an occluder chamber 275. The occluder 270 is operable to inflate between at least contracted position 277 and dilated position 279. The occluder 270 is illustrated in the contracted position 77 in FIG. 4H.

As illustrated in FIG. 4I, the occluder 270 is inflated into a dilated position 279 to retain the occluding agent 100 within the vein segment 400. As illustrated, the occluder 270 in the dilated position 279 is positioned such that, when dilated, portions of the occluder outer surface 272 are biased against the occluding agent 100 to compress the occluding agent 100. Portions of the occluder outer surface 272 are also biased against the vein inner surface 404 to lock the occluder 270 into place. In some implementations, a gas may be communicated into the occluder chamber 275 to inflate the occluder 270. In other implementations, a liquid such as saline solution may be communicated into the occluder chamber 275 to inflate the occluder 270. In still other implementations, the occluding agent 100 may communicated into the occluder chamber 275 to inflate the occluder 270.

FIG. 4J shows the occluding agent 100 interposed between the occluder 170 and the occluder 270 in the vein segment 400, after the inner tube distal end 58 has been detached from the occluder 270, the barrier 60 has been deflated into a collapsed position 67, and the catheter 10 including the inner tube 50 and the outer tube 40 has been withdrawn. The occluder 170 and the occluder 270 lock the occluding agent 100 in the solid state into the vein segment 400 to form the structural member 500. In some implementations, the occluding agent 100 may adhere to portions of the occluder outer surface 172 and to portions of the occluder outer surface 272 such that the occluder 170, the occluding agent 100, and the occluder 270 form a generally unitary structural member 500 in the vein segment 400 between the vein segment proximal end 406 and the vein segment distal end 408. Portions of the occluder 270 define a structural member proximal end 506 and portions of the occluder 170 define a structural member distal end 508, as illustrated.

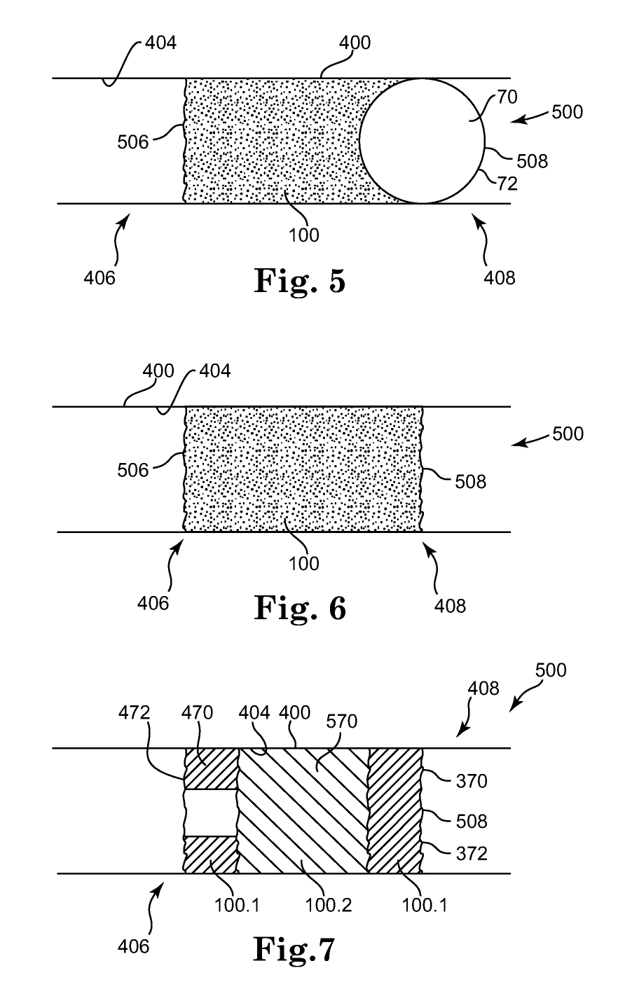

FIG. 5 illustrates an implementation of the structural member 500 that includes the occluder 70 in combination with the occluding agent 100. In this implementation, the occluder 70 defines the structural member distal end 508 and portions of the occluding agent 100 in the solid state define the structural member proximal end 506.

FIG. 6 illustrates another implementation of the structural member 500 that includes the occluding agent 100, with the structural member distal end 508 and the structural member proximal end 506 defined by portions of the occluding agent 100 in the solid state. In implementations wherein the occluding agent 100 rapidly solidifies from the liquid state to the solid state, the occluder(s) 70 may not be needed to lock the occluding agent 100 into position within the vein segment 400.

As illustrated in FIG. 7, occlusions may be formed from fast-setting or fast-curing occluding agent, or by materials such as sponges. Where fast-setting occluding agent is used, for example, the catheter 10 may be positioned with the inner tube 50 extended. However, instead of providing the barrier 60 and the occluder 70, ports may be substituted for dispensing an occluding agent 101.1 that is designed for rapid solidification from a fluid state to a solid state. The rapidly setting occluding agent 101.1 may be dispensed into the vein segment 400 from the ports to form terminal occlusion 370 and occlusion 470, and a second occluding agent 100.2 may be delivered into the vein segment 400 between the terminal occlusions 370 and 470 to form a primary occlusion 570. The occlusions 370, 470 and 570 in this implementation are all substantially uniformly solid. In various implementations, different types of occluding agents may be used for the occluding agents 101.1 and 101.2, and even for forming the occlusions 370 and 470 if desired, with the various types of occluding agent 100 being selected to produce specific desired therapeutic effects. The materials used in the catheter should be selected to minimize bonding between the cured occluding agents and the catheter, to allow for withdrawal of the catheter. A suitable rapid-setting occluding agent is CoSeal® surgical sealant, which is available from Angiotech Pharmaceuticals of Vancouver, British Columbia, Canada, and Baxter Healthcare Corporation of Fremont, Calif.

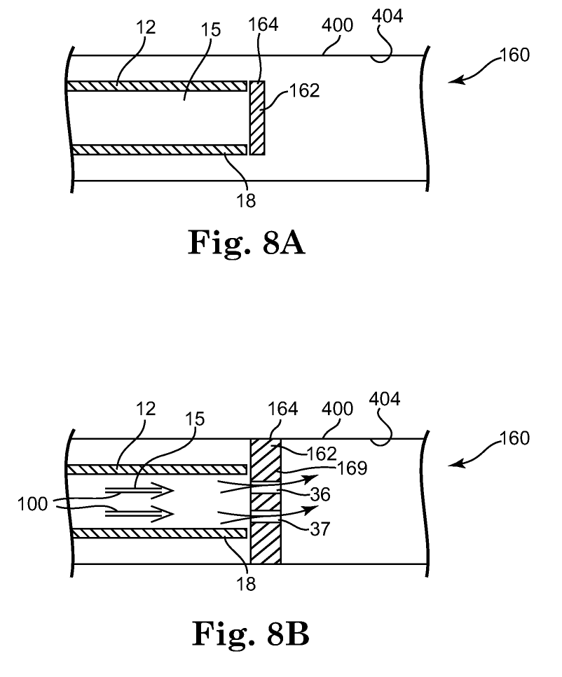

FIGS. 8A and 8B show a catheter 160 that has an alternative implementation 162 of the barrier 60. FIG. 8A shows the barrier 162 disposed about the distal end of the catheter tube 12 of the catheter 160 in a collapsed condition. When in a collapsed condition, the barrier 162 may be positioned in the vein segment 400, as illustrated in FIG. 8A. The barrier 162, as illustrated in FIG. 8B, may be transformed into an expanded condition to bias portions of the barrier outer surface 164 circumferentially against the vein inner surface 404. As illustrated in FIG. 8B, the barrier 162 in the expanded position is deployed generally across the vein segment in the form of a diaphragm to occlude the vein segment 400. A first orifice 36 and a second orifice 37 are opened in the expanded barrier 162 so that occluding agent 100 in the liquid state may flow through the lumen 15 of the catheter 10, through the first orifice 36 and the second orifice 37, and into the vein segment 400 distal of the barrier 162. When the occluding agent 100 solidifies sufficiently, the barrier 162, in this illustrated implementation, may be transformed into the contracted position and withdrawn with the catheter tube 12 from the vein segment 400. In other implementations, the barrier 162 may be designed to be released from the distal end of the catheter tube 12 to remain in the vein segment after the catheter tube 12 is withdrawn. Illustratively, for this example the barrier 162 may be a sponge. In other implementations, the barrier 162 may be configured in other ways to be attached to the catheter tube 12 generally proximate the distal end to occlude the vein segment and to allow the delivery of occluding agent 100 into the vein segment 400 distal of the barrier 160.

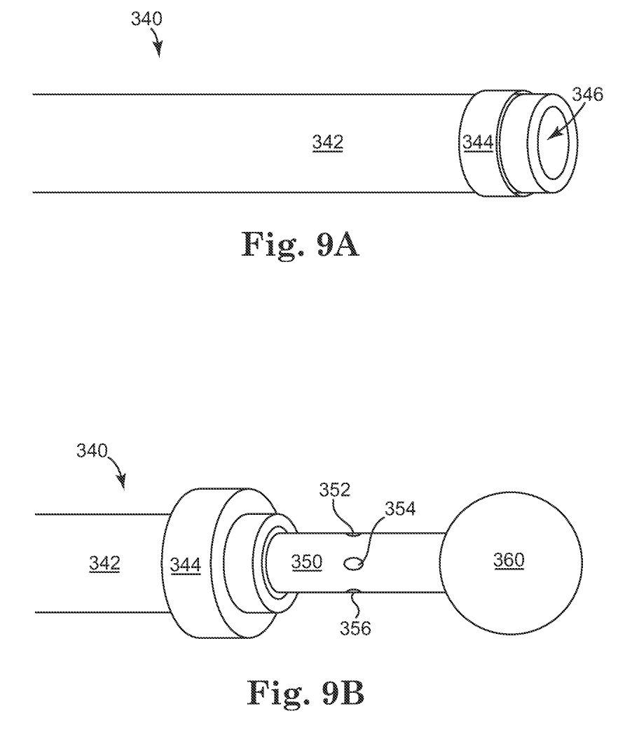

A suitable catheter 340 for injecting occluding agent into the cardiac venous system is shown in various operating conditions in FIGS. 9A and 9B. FIG. 9A shows the catheter 340 in a condition suitable for being advanced through a cardiac vein, and FIG. 9B shows the catheter 340 in a deployed condition for defining a segment of a cardiac vein into which the occluding agent may be introduced. This illustrated implementation of the catheter 340 includes an outer tube 342 and an inner tube 350. The outer tube 342 defines a lumen 346 for communicating fluid and/or for communicating the inner tube 350 into the vein. The outer tube 342 and the inner tube 350 may be made from a variety of materials alone or in combination, including metals such as, for example, stainless steel or nitinol, and polymers such as polyethylene, nylon, and polyimide, among others.

The catheter 340 includes an expandable barrier 344, shown collapsed in FIG. 9A and expanded in FIG. 9B. The barrier 344 extends circumferentially around the outer tube 342 generally proximate the distal end thereof to block blood flow through the vein. The barrier 344 may be constructed of a variety of different materials, including, for example, Nylon, PEEK, and Pebax, among others. The outer tube 342 includes a lumen (not shown) to transmit fluid to/from the barrier 344 in order to inflate/deflate the barrier 344. With the barrier 344 in its collapsed position, the catheter 340 may be moved and positioned as desired within the cardiac venous system. When the catheter 340 is properly positioned, the barrier 344 is expanded to engage the wall of the vein, thereby stabilizing the distal end of the catheter 340, blocking blood flow through the vein, and establishing one end of the segment into which the occluding agent is to be introduced.

The inner tube 350 is slidably received within the lumen 346 for placing an occluder 360 within the vein in a spaced-apart relationship with the barrier 344. The occluder 360, which is removably disposed at the distal end of the inner tube 350, is passed through the lumen of the outer tube 342 in a collapsed condition, is advanced through the vein a desired distance from the distal end of the outer tube 342, and is expanded to engage the wall of the vein for establishing the other end of the segment into which the occluding agent is to be introduced. Although shown in FIG. 9B as being generally spherical as deployed, the occluder 360 may be any desired shape.

The inner tube 350 includes two lumen (not shown). One of these lumen is for transmitting fluid to/from the occluder 360 where the occluder 360 is designed to be inflatable and releasable, or inflatable and deflatable, or for containing a wire for mechanically releasing the occluder 360 where the occluder is designed to expand upon release, such as a sponge. Where the occluder is designed to expand upon release, the release wire and associated lumen may be eliminated if the release mechanism is triggered by the pressure of occluding agent injectate in the other lumen. The other lumen is for communicating fluid with ports 352, 354 and 356 (illustratively three ports are shown) on the inner tube 350. The ports 352, 354 and 356 are for introducing occluding agent into the between the occluder 360 and the barrier 344, and may be used to suction blood from the volume if desired.

To place occluding agent within a desired segment of the cardiac venous system, the catheter 340 as shown in FIG. 9A is advanced to a desired position within the cardiac venous system. The barrier 344 is expanded to stabilize the distal end of the catheter 340, to define one end of the segment, and to block the flow of blood to the heart from the segment. The inner tube 350 is advanced a desired distance into the vein from the distal end of the outer tube 342, and the occluder 360 is expanded to define the other end of the segment. Occluding agent is introduced into the segment through the ports 352, 354 and 356. The occluding agent is permitted to cure, and the inner tube 350 is retracted and removed from the catheter 340. The occluder 360 may be collapsed for withdrawal, or may be detached prior introduction of occluding agent, or may automatically detach as retraction of the inner tube 350 begins. The barrier 344 is collapsed and the catheter 340 is removed from the site. Optionally, a second occluder similar to the occluder 360 may be placed at the other end of the segment before the catheter 340 is removed.

Radiopaque materials may be disposed proximate the distal end of the outer tube 342 and/or the distal end of the inner tube 350 to facilitate or confirm proper placement of the distal end of the catheter 340 within the desired segment of the cardiac venous system.

The proximal ends (not shown) of the outer tube 342 and the inner tube 350 extend from the proximal end of the catheter 340 and are connected to a handle (not shown) to allow the a physician to control the various functions performed by the catheter 340.

Methods for forming a structural member 500 in the vein segment 400 to reinforce the myocardium include delivering the occluding agent 100 into the vein segment 400, the occluding agent 100 solidifying within the vein segment 400 thereby forming the structural member 500. Treating a particular cardiac condition by selecting one or more vein segments 400, delivering the occluding agent 100 into the one or more vein segments 400 thereby forming one or more structural members 500 within the one or more vein segments 400 may be included in the methods. The catheter 10 may define one or more lumen 15 for delivering the occluding agent 100 into the vein segment 400. The methods, in various implementations, may include positioning at least one occluder 70 within the vein thereby occluding the vein segment 400 and retaining the occluding agent 100 within the vein segment 400. Positioning at least one occluder 70 using the catheter 10 may be included in the methods. The methods may include disposing the occluding agent 100 within the vein segment 400 between an occluder 170 and an occluder 270. Various implementations of the methods may include the occluder 170, the occluding agent 100, and the occluder 270 defining the structural member 500. Including a therapeutic material in the occluding agent 100 may be part of the methods in various aspects.

Delivering the occluding agent 100 into the vein segment 400 by a particular implementation of the catheter 10 may proceed in the following manner. The method may be initiated by placing the outer tube distal end 48 within the vein segment 400 by navigating the catheter 10 through various bodily lumen. Transforming the barrier 60 (FIGS. 4A-4I) from the collapsed position 67 into the expanded position 69, or the barrier 162 from the collapsed position (FIG. 8A) into the expanded position 169 (FIG. 8B), thereby biasing the barrier outer surface (62,164) against the vein inner surface 404, occluding the vein, and anchoring the outer tube 40 into position within the vein segment 400 may be part of the methods. The methods may include passing the inner tube 50 through the outer lumen 45 and extending the inner tube distal end 58 through the outer aperture 80 into the vein segment 400. Positioning the occluder 170 within the vein segment 400 by manipulating the inner tube 50, and biasing portions of the occluder outer surface 172 against the vein inner surface 404 by inflating the occluder 170 from the contracted position 177 into the dilated position 179 thereby occluding the vein and locking the occluder 170 into position within the vein segment 400 may be included in the methods.

The methods may include removing the blood from the vein segment 400. The methods, in various aspects, may include inflating the occluder 170 from the contracted position 177 into the dilated position 179 and pushing the occluder 170 in the dilated position distally to push blood distally from the vein segment 400. In various aspects, the methods may include withdrawing blood from the vein segment 400 through annular lumen 255 and/or through inner lumen 55.

The methods, in various aspects, may include delivering the occluding agent 100 into the vein segment 400 through the outer aperture 80 via the annular lumen 255, the occluder 170 and the barrier 60 holding the occluding agent 100 within the vein segment 400. In other aspects, the methods may include detaching the inner tube distal end 58 from the occluder 170, and delivering the occluding agent 100 via the inner lumen 55 through the inner aperture 90 into the vein segment 400. In still other aspects, the methods may include detaching the inner tube 50 from the occluder 170, withdrawing the inner tube 50 from the outer lumen 45, and delivering the occluding agent 100 into the vein segment 400 through the outer aperture 80 via the outer lumen 45.

The methods may include detaching the inner tube distal end 58 from the occluder 170 and withdrawing the inner tube distal end 58 may be included in the methods, the barrier 60 remaining in the expanded position 69 thereby securing the occluding agent 100 in the vein segment 400. In various aspects, the inner tube 50 may be substantially or entirely withdrawn from the outer lumen 45.

The methods may proceed by withdrawing the outer tube distal end 48 somewhat in the proximal direction with the barrier 60 in the expanded position 69. Passing the inner tube 50 through the outer lumen 45 and extending the inner tube distal end 58 forth from the outer aperture 80 into the vein segment 400, and inflating a occluder 270 secured to the inner tube distal end 58 thereby retaining the occluding agent 100 between the occluder 170 and the occluder 270 may be steps in the various methods. Compressing the occluding agent 100 within the vein segment 400 by dilating the occluder 270 and locking the occluder 270 into position in the vein segment 400 by biasing portions of the occluder outer surface 272 against the vein inner surface 404 may be part of the methods. The methods may include detaching the inner tube distal end 58 from the occluder 270, deflating the barrier 60 into the collapsed position 67, and withdrawing the catheter 10 including the inner tube 50 and the outer tube 40 thereby locking the occluding agent 100 into the vein segment 400 by interposing the occluding agent 100 between the occluder 170 and the occluder 270.

In a variation of the catheters 10 and 340 and the methods of operating them, the inner tubes 50 and 350 may be fixed or have very limited slidable motion relative to the outer tubes 40 and 342. With reference to the catheter 340 (FIGS. 9A and 9B), for example, the distal end of the inner tube 350 may be at or project from the distal end of the outer tube 342 even when the catheter 340 is configured for being advanced through a cardiac vein. If projecting from the distal end of the outer tube 342, the amount of projection may be small, or may be on the order of the intended length of the vein segment to be occluded. The expandable barrier 344 and the occluder 360 are collapsed while the catheter 340 is being advanced through the cardiac vein. When the catheter 340 is properly positioned, the barrier 344 is expanded to engage the wall of the vein, thereby stabilizing the distal end of the catheter 340, blocking blood flow through the vein, and establishing one end of the segment into which the occluding agent is to be introduced. If the inner tube 350 is in this variation slidable with respect to the outer tube 342, it may be extended or retracted as needed to establish the proper segment length before the occluder 360 is expanded. If the inner tube 350 is in this variation fixed with respect to the outer tube 342, the occluder 360 is expanded, which may but need not be done concurrently with expansion of the barrier 344. In either case, expanding the occluder 360 establishes the other end of the segment into which the occluding agent is to be introduced. To complete the occlusion, the occluding agent is introduced, the inner tube 350 is detached from the occluder 360 and either left projecting from the distal end of the outer tube 342 or drawn into the outer tube 342 either completely or partially, the barrier 344 is collapsed, and the catheter 340 is removed.

The various exemplary implementations described herein are illustrative of the invention. Variations and modifications of these implementations are possible, and practical alternatives to and equivalents of the various elements of the embodiments are contemplated. These and other variations and modifications of the implementations disclosed herein may be made without departing from the scope and spirit of the invention, as set forth in the following claims.

1. A catheter for establishing an occlusion within a cardiac vein, comprising:
an elongated catheter body having a distal end and a proximal end and comprising an injectate lumen extending longitudinally within the catheter body;
a barrier disposed about a periphery of the catheter body in proximity to the distal end thereof and about the injectate lumen, the barrier being controllably transformable between a collapsed position for movement of the catheter body within the cardiac vein, and an expanded position for occluding the cardiac vein in cooperation with the catheter body; and
an injectate port disposed in the catheter body distally of the barrier, the injectate lumen being in fluid communication with the injectate port.

2. The catheter of claim 1 wherein the catheter body comprises:
an outer tube, the barrier being circumferentially disposed upon the outer tube proximate a distal end of the outer tube; and
an inner tube;;
further comprising an occluder disposed at a distal end of the inner tube, the occluder being controllably transformable between a collapsed position, and an expanded position for occluding the cardiac vein.

3. The catheter of claim 2 wherein:
the inner tube projects from the outer tube and has a distal end extending beyond the distal end of the outer tube; and
the occluder is controllably transformable between a collapsed position for accommodating movement of the catheter body within the cardiac vein, and the expanded position.

4. The catheter of claim 2wherein:
the inner tube projects from the distal end of the outer tube and is slidably disposed within the outer tube for movement therein, the inner tube having a distal end extending beyond the distal end of the outer tube; and
the occluder is controllably transformable between a collapsed position for accommodating movement of the catheter body within the cardiac vein, and an expanded position for occluding the cardiac vein.

5. The catheter of claim 1 wherein:
the inner tube is slidably disposed within the outer tube for movement therein, the inner tube having a distal end that is extendable beyond the distal end of the outer tube;
further comprising an occluder disposed at the distal end of the inner tube, the occluder being controllably transformable between a collapsed position for movement within the outer tube in cooperation with the inner tube, and an expanded position when the inner tube is extended beyond the distal end of the outer tube for occluding the cardiac vein.

6. The catheter of claim 2 further comprising a coupler for releasably coupling the occluder to the inner tube.

7. The catheter of claim 2 wherein:
the occluder comprises an expandable interior wall defining an interior space; and
the inner tube comprises a lumen in fluid communication with the interior space of the occluder for inflating the occluder into the expanded position.

8. The catheter of claim 2 wherein the occluder comprises an expandable sponge.

9. The catheter of claim 2 wherein:
the injectate port is disposed proximate the distal end of the inner tube; and
the injectate lumen is contained within the inner tube.

10. The catheter of claim 1 further comprising an occluding agent source in fluid communication with the injectate lumen.

11. The catheter of claim 1 wherein the catheter body comprises an additional injectate lumen extending within the catheter body, the barrier being disposed about the additional injectate lumen, and the injectate port being in fluid communication with the additional injectate lumen.

12. The catheter of claim 11 further comprising a source of a first occluding agent component in fluid communication with the injectate lumen, and a source of a second occluding agent source in fluid communication with the additional injectate lumen.

13. The catheter of claim 1 further comprising:
a first additional injectate port disposed in the catheter body distally of the barrier;
wherein the catheter body further comprises a first additional injectate lumen extending longitudinally within the catheter body, the barrier being disposed about the first additional injectate lumen, and the first additional injectate lumen being in fluid communication with the first additional injectate port;

14. The catheter of claim 13 further comprising a source of a first occluding agent component in fluid communication with the injectate lumen, and a source of a second occluding agent component in fluid communication with the first additional injectate lumen.

15. The catheter of claim 13 further comprising:
a second additional injectate port disposed in the catheter body distally of the barrier;
wherein the catheter body further comprises a second additional injectate lumen extending longitudinally within the catheter body, the barrier being disposed about the second additional injectate lumen, and the second additional injectate lumen being in fluid communication with the second additional injectate port;

16. The catheter of claim 2 wherein the injectate port comprises an annular aperture disposed between the inner tube and the outer tube.

17. The catheter of claim 2 wherein the injectate port comprises an aperture disposed in a wall of the inner tube.

18. The catheter of claim 1 wherein the catheter body comprises:
an outer tube, the barrier being circumferentially disposed upon the outer tube proximate a distal end of the outer tube; and
an inner tube slidably disposed within the outer tube for movement therein, the inner tube having a distal end that is extendable beyond the distal end of the outer tube;
further comprising an occluder releasably disposed at the distal end of the inner tube, the occluder being controllably transformable between a collapsed position for movement within the outer tube in cooperation with the inner tube, and an expanded position when the inner tube is extended beyond the distal end of the outer tube for occluding the cardiac vein, and the occluder comprising an expandable interior wall defining an interior space; and
a coupler for releasably coupling the occluder to the inner tube;
wherein the outer tube comprises a barrier lumen in fluid communication with the barrier for inflating the barrier into the expanded position and deflating the barrier into the collapsed position; and
wherein the inner tube comprises an occluder lumen in fluid communication with the occluder interior space for inflating the occluder into the expanded position.

19. A catheter for establishing an occlusion within a cardiac vein, comprising:
means for advancing a distal end of a catheter to a site within the cardiac vein;
means for establishing a first venous occlusion about the catheter near a distal end thereof to occlude the cardiac vein at the site, in cooperation with the catheter;
means for introducing an occluding agent into the cardiac vein at the site to form at the site a second venous occlusion generally contiguous to the first venous occlusion; and
means for withdrawing the distal end of the catheter from the site following the occluding agent introducing step.

20. The catheter of claim 19 further comprising:
means for establishing, in cooperation with the catheter, a third venous occlusion about the catheter near the distal end thereof and spaced way from the first venous occlusion to define a cardiac vein segment;
wherein the introducing means comprises means for introducing the occluding agent into the cardiac vein segment to form the second venous occlusion generally contiguous to the first and third venous occlusions.

21. A method for establishing an occlusion within a segment of a cardiac vein, comprising:
advancing a distal end of a catheter to a site within the cardiac vein;
establishing a first venous occlusion about the catheter in proximity to a distal end thereof to occlude the cardiac vein at the site, in cooperation with the catheter;
introducing an occluding agent into the cardiac vein at the site to form at the site a second venous occlusion generally contiguous to the first venous occlusion; and
withdrawing the distal end of the catheter from the site following the occluding agent introducing step.

22. The method of claim 21 wherein the first venous occlusion establishing step comprises expanding a barrier disposed circumferentially about the catheter in proximity to the distal end thereof.

23. The method of claim 21 wherein the first venous occlusion establishing step comprises dispensing a fast setting occluding agent from the catheter to form an occlusion disposed about the catheter in proximity to the distal end thereof.

24. The method of claim 21 further comprising:
prior to the introducing step, establishing a third venous occlusion at a location within the cardiac vein spaced away from the first venous occlusion;
wherein the introducing step further comprises introducing the occluding agent into the cardiac vein between the first venous occlusion and the third venous occlusion, the second venous occlusion being generally contiguous to the first and third venous occlusions.

25. The method of claim 24 wherein the third venous occlusion establishing step comprises:
positioning a fluid-expandable occluder at the spaced-away location; and
introducing a fluid from the catheter into the occluder to expand the occluder and occlude the vein.

26. The method of claim 24 wherein the third venous occlusion establishing step comprises releasing a self-expanding occluder from the distal end of the catheter at the spaced-away location to occlude the vein.

27. The method of claim 24 wherein the third venous occlusion establishing step comprises dispensing a fast setting occluding agent from the distal end of the catheter at the spaced-away location to occlude the vein.

28. A method for establishing an occlusion within a cardiac vein, comprising:
positioning a distal end of a catheter outer tube within the vein;
expanding a barrier disposed about the catheter outer tube near the distal end thereof for occluding the vein at a first location with an expanded barrier, in cooperation with the catheter;
positioning a distal end of a catheter inner tube within the vein and spaced apart from the barrier, the distal end of the catheter inner tube having an occluder coupled thereto;
expanding the occluder within the vein for occluding the vein with an expanded occluder at a second location spaced-away from the first location;
introducing occluding agent from the catheter into the vein between the expanded barrier at the first location and the expanded occluder at the second location;
releasing the expanded occluder from the distal end of the inner tube;
collapsing the barrier; and
withdrawing the catheter from the vein.

29. The method of claim 28 further wherein:
the catheter inner tube positioning step comprises advancing the catheter inner tube and the occluder through a lumen of the catheter outer tube and into the vein; and
the catheter withdrawing step comprises:
withdrawing the catheter inner tube through the catheter outer tube; and
withdrawing the catheter outer tube from the vein.

30. A heart prosthetic for treating a heart in a diseased condition, comprising:
a first occlusion of a first composition disposed within a part of a cardiac vein; and
a second occlusion of a second composition different than the first composition disposed within a part of the cardiac vein contiguous to the first occlusion;
the first and second occlusions being essentially in a solid state for provide structural support to the heart.

31. The heart prosthetic of claim 30 wherein the first occlusion is an expanded occluder and the second occlusion is formed of an occluding agent.

32. The heart prosthetic of claim 30 wherein the first occlusion is formed of a fast-setting occluding agent, and the second occlusion is formed of an occluding agent different than the fast-setting occluding agent.

33. The heart prosthetic of claim 30 further comprising:
a third occlusion of a third composition different than the second composition disposed within a part of the cardiac vein contiguous to the second occlusion;
the third occlusion being essentially in a solid state for provide structural support to the heart.

34. The heart prosthetic of claim 30 wherein the first occlusion comprises an expanded occluder, the second occlusion is formed of an occluding agent, and the third occlusion comprises an expanded occluder.

35. The heart prosthetic of claim 30 wherein the first occlusion is formed of a first fast-setting occluding agent, the third occlusion is formed of a second fast-setting occluding agent, and the second occlusion is formed of an occluding agent different than the first and second fast-setting occluding agents.

36. The heart prosthetic of claim 35 wherein the first and second fast-setting occluding agents are identical.

37. A kit comprising:
a source of an injectable occluding agent; and
a catheter comprising:
an elongated catheter body having a distal end and a proximal end and comprising an injectate lumen extending longitudinally within the catheter body, and a barrier disposed about a periphery of the catheter body in proximity to the distal end thereof and about the injectate lumen, the barrier being controllably transformable between a collapsed position for movement of the catheter body within the cardiac vein, and an expanded position for occluding the cardiac vein in cooperation with the catheter body;
a coupler in fluid communication with the injectate lumen, the occluding agent source being adapted for coupling to the first coupler; and
an injectate port disposed in the catheter body distally of the barrier, the injectate lumen being in fluid communication with the injectate port.

38. The kit of claim 37 wherein the catheter body further comprises:
an outer tube, the barrier being circumferentially disposed upon the outer tube proximate a distal end of the outer tube; and
an inner tube slidably disposed within the outer tube for movement therein, the inner tube having a distal end that is extendable beyond the distal end of the outer tube;
further comprising an occluder disposed at the distal end of the inner tube, the occluder being controllably transformable between a collapsed position, and an expanded position when the inner tube is extended beyond the distal end of the outer tube for occluding the cardiac vein.

39. The kit of claim 37 wherein:
the occluding agent is a multiple-component agent;
the source comprises a first source section for a first component of the multiple-component agent, and a second source section for a second component of the multiple-component agent; and
the catheter body further comprises:
an additional injectate lumen extending longitudinally within the catheter body, the barrier being disposed about the additional injectate lumen; and
an additional coupler in fluid communication with the additional injectate lumen, the first source section being adapted for coupling to the coupler, and the second source section being adapted for coupling to the additional coupler;
wherein the additional injectate lumen is in fluid communication with the injectate port.

40. The kit of claim 37 wherein:
the occluding agent is a multiple-component agent;
the source comprises a first source section for a first component of the multiple-component agent, and a second source section for a second component of the multiple-component agent; and
the catheter body further comprises:
an additional injectate lumen extending longitudinally within the catheter body, the barrier being disposed about the additional injectate lumen;
an additional coupler in fluid communication with the additional injectate lumen, the first source section being adapted for coupling to the coupler, and the second source section being adapted for coupling to the additional coupler; and
an additional injectate port disposed in the catheter body distally of the barrier, the additional injectate lumen being in fluid communication with the additional injectate port.
